(19) United States
(12) Patent Application Publication
(10) Pub. No.: US 2013/0287714 A1
(43) Pub. Date: Oct. 31, 2013

(54) COSMETIC AND/OR DERMATOLOGICAL PREPARATIONS CONTAINING SNOW ALGAE EXTRACT

(71) Applicants: Sven Gohla, Stafa (DE); Daniel Stangl, Meggen (CH); Claudia Mundt, Basel (CH)

(72) Inventors: Sven Gohla, Stafa (DE); Daniel Stangl, Meggen (CH); Claudia Mundt, Basel (CH)

(21) Appl. No.: 13/883,415

(22) PCT Filed: Nov. 12, 2010
(86) PCT No.: PCT/EP2010/067341

§ 371 Date: Jul. 17, 2013

(51) Int. Cl. A61K 8/97; A61Q 19/08; A61Q 17/04

(52) U.S. Cl. CPC A61K 8/975; A61Q 17/04; A61Q 19/08
USPC 424/59; 424/195.17

(57) ABSTRACT

The present invention describes cosmetic or dermatological preparations which contain snow algae extracts and one or several substances which are suitable for skin protection.

The present invention describes cosmetic or dermatological preparations which comprise snow algae extracts for achieving skin protection.

Cosmetic or dermatological preparations which are aimed at protecting the skin care not novel in principle. In the past, there has already been various compositions in cosmetics and in dermatology whose task was to protect skin, in particular human skin. Nevertheless, there continues to be the need to improve or to modify skin protection because the environmental conditions and also living conditions and habits of users are changing.

Thus, for example, the increased emission of CO₂ into the atmosphere brings about climate change which is linked to an increase in temperature. This has effects on the human skin, which requires increased protection and also modified protection.

In the course of progress, there are contaminations of the environment, in particular of the air. These contaminations can be gaseous in the form of carbon dioxide, methane, halogenated and partly halogenated hydrocarbons and other gases, or particulate, e.g. in the form of soot particles. All of these contaminations come into contact with the skin and can cause irritations ranging to severe disorders. This too renders improved and/or modified skin protection necessary.

Altered living conditions lead to many people, especially in western countries, spending long periods of the day in closed rooms. At cold times of the year, these rooms are heated and the room air is consequently in most cases very dry. The skin reacts to this by drying out. Improved skin protection is necessary.

Snow algae extracts are extracts of snow algae which can be obtained from Chlamydomonas spec. These extracts can be obtained from snow algae which are cultured using the method of a two-phase cryocultivation. After the cultivation, a whole extract can be produced by means of high-pressure homogenization using empty liposomes. Simultaneous drying and application of the extract to a support material by means of fluidized-bed drying can conclude the production of the extract. The support material can consist for example of isomalt, lecithin, sodium benzoate, lactic acid and water in various weight fractions. The snow algae extract can be present for example in powder form and have the following composition:

| | |
|---|---|
| Isomalt | 92 |
| Water | 5.34 |
| Chlamydomonas extract | 2 |
| Sodium benzoate | 0.3 |
| Lactic acid | 0.2 |
| Lecithin | 0.16, |

all data in % by weight.

The snow algae extracts thus obtained are available for example from Mibelle and carry the name Mibexol.

Snow algae are freshwater algae which belong to the Chlamydomonas genus. Nowadays, about 350 species of snow algae are known, with Chlamydomonas nivalis, a single-celled alga, being particularly widespread. Snow algae occur on snow and glacier surfaces in polar and alpine regions. Their mass reproduction is responsible for the green and red coloration of snow; e.g. blood snow. During the winter, the algae rest in the form of spores, often red in color, below the snow. In spring, the spores produce green, UV-light-sensitive algae which migrate to the surface of the snow. There, with the help of photosynthesis, the necessary energy is obtained and reproduction begins. Nutrient limitation results again in sporulation, i.e. the formation of red spores. The red coloration stems from an incorporation of carotenoids (astaxanthin) for UV protection. The living conditions of snow algae are extreme, the temperatures are low, the UV radiation is strong and nutrients are scarce. The snow algae therefore have to live on water, CO₂, sunlight and minerals. The optimum growth temperatures are 0 to +5° C. In order to be able to survive under these conditions, a series of adaptations can be found among the snow algae, detectable by the presence of various secondary metabolites, such as pigments (including carotenoids, e.g. astaxanthin), biopolymers (gels), antifreeze proteins, antifreeze glycoproteins and osmotically effective amino acids and sugars.

The use of Chlamydomonas extracts in cosmetic preparations has already been described on occasion.

EP 1437124 B1 describes a composition for treating acne. An algae extract which is obtained from the alga Chlamydomonas reinharhardtii is used together with other substances.

The abstract of JP 2010013416 A discloses an algae extract of an alga which belongs to the Chlamydomonas genus and has a hair growth promoting effect.

The extract of an alga which belongs to the Chlamydomonas genus is described in the abstract of JP 2010013413A. This extract has a whitening effect.

The abstract of JP 2006028412 describes an extract of Chlamydomonas algae, preferably Chlamydomonas strain W80, which is used as an antioxidant.

An antioxidant is likewise disclosed in the abstract of JP 2002114703A. This antioxidant can be obtained inter alia from Chlamydomonas species.

The teachings in the prior art do not explicitly disclose the use of snow algae extracts nor their particular properties with regard to use in cosmetics or dermatology.

It is therefore an object of the present invention to provide cosmetic or dermatological preparations which ensure skin protection, in particular for the human skin. This is to be accomplished especially with regard to the changed environmental conditions. Moreover, the preparations should be simple and easy to use.

This object is achieved by cosmetic and/or dermatological preparations which comprise snow algae extracts and are used for protecting the skin.

Cosmetic preparations comprising snow algae extract for protecting skin, in particular human skin, are in accordance with the invention.

According to the invention, preference is given to cosmetic preparations for protecting skin, in particular human skin, which comprise, besides snow algae extract, isomalt, lecithin, sodium benzoate, lactic acid and water.

The cosmetic and/or dermatological preparations have a concentration of snow algae extracts of from 0.0001 to 99% by weight, preferably 0.0005 to 20% by weight, particularly preferably 0.001 to 10% by weight, based on the total weight of the preparation.

The cosmetic or dermatological preparations comprising snow algae extract can furthermore comprise one or more of the following substances:
a. collagen and/or a derivative
b. chitosan and/or acetylated chitosan with a degree of acetylation of about 50%
c. at least one glycosylaminoglycan and/or a derivative and at least one, preferably both, of the following ingredients:
d. at least one peptide which promotes cell growth,
e. a composition comprising
   i. glycoprotein 1
   ii. glycoprotein 2
   iii. ginseng extract and
   iv. horsetail extract
      for protecting skin.

According to the invention, preference is given to cosmetic preparations for protecting skin, in particular human skin, which, besides snow algae extract and one and/or more of the aforementioned substances, also comprise isomalt, lecithin, sodium benzoate, lactic acid and water.

The above preparations comprise snow algae extract and one and/or more of the above substances together in a concentration of from 0.0001 to 99% by weight, preferably 0.0005 to 20% by weight, particularly preferably 0.001 to 10% by weight, based on the total weight of the preparation.

Furthermore, the use of snow algae extract in cosmetic preparations for protecting skin, in particular for protecting against modern stresses, is in accordance with the invention. Also in accordance with the invention is the use of snow algae extract and isomalt, lecithin, sodium benzoate, lactic acid and water in cosmetic preparations for protecting skin, in particular for protecting against modern stresses.

Also in accordance with the invention is the use of snow algae extracts for protecting skin against the effect of free radicals. Moreover in accordance with the invention is the use of snow algae extract for protecting skin against effects of UV light. Also in accordance with the invention is the incorporation of snow algae extract into products which serve for UV protection of the skin. An intensification of the UV protection can be achieved.

Also in accordance with the invention is the use of snow algae extract for protecting skin for increasing the longevity of skin cells, as a result of which intrinsic and/or extrinsic skin ageing is counteracted.

Also in accordance with the invention is a method for protecting skin, in particular human skin, where cosmetic and/or dermatological preparations comprising snow algae extract are applied to the skin.

Also in accordance with the invention is a method for protecting skin, in particular human skin, where cosmetic and/or dermatological preparations comprising snow algae extract and isomalt, lecithin, sodium benzoate, lactic acid and water are applied to the skin.

Also in accordance with the invention is the use of snow algae extract and one or more of the following substances:
a. collagen and/or a derivative
b. chitosan and/or acetylated chitosan with a degree of acetylation of about 50%
c. at least one glycosylaminoglycan and/or a derivative and at least one, preferably both, of the following ingredients:
d. at least one peptide which promotes cell growth,
e. a composition comprising
   i. glycoprotein 1
   ii. glycoprotein 2
   iii. ginseng extract and
   iv. horsetail extract
      in cosmetic and/or dermatological preparations for protecting skin.

Moreover in accordance with the invention is the use of snow algae extract and one or more of the substances listed above together with isomalt, lecithin, sodium benzoate, lactic acid and water for protecting skin.

Furthermore in accordance with the invention is the use of snow algae extract and one or more of the substances listed above for protecting skin against modern stresses, where protection against the effects of free radicals and UV light is also included. Also further in accordance with the invention is the incorporation of snow algae extract and one or more of the substances listed above into products which serve for the UV protection of skin. An intensification of the UV protection can be achieved.

Moreover, the use of snow algae extract and one or more of the substances listed above for protecting skin is also inventive, the protection being effected by increasing the longevity of skin cells. According to the invention in particular is protection of skin by snow algae extracts and one or more of the substances listed above against intrinsic and/or extrinsic skin ageing. This can be brought about by increasing the longevity of skin cells.

Also in accordance with the invention is a method for protecting skin, in particular human skin, where cosmetic and/or dermatological preparations comprising snow algae extract and one or more of the substances listed above are applied to the skin. Also in accordance with the invention is a method for protecting skin, in particular human skin, where cosmetic and/or dermatological preparations comprising snow algae extract and one or more of the substances listed above together with isomalt, lecithin, sodium benzoate, lactic acid and water are applied to the skin.

The preparations according to the invention are also suitable for providing protection for the skin, in particular human skin, which protects the skin against the effects of stress. These effects of stress can be caused by free radicals or compounds which come into ever more frequent contact with the human skin in the course of increasing environmental pollution.

The preparations according to the invention are also suitable for increasing the longevity of skin cells and therefore of the skin. Longevity of the skin cells gives the skin a plus in terms of life. This is desirable in order to counteract skin ageing caused by intrinsic processes and/or extrinsic factors.

In accordance with the invention in particular is the incorporation of snow algae extract and/or snow algae extract and one or more of the following substances:
a. collagen and/or a derivative
b. chitosan and/or acetylated chitosan with a degree of acetylation of about 50%
c. at least one glycosylaminoglycan and/or a derivative and at least one, preferably both, of the following ingredients:
d. at least one peptide which promotes cell growth,
e. a composition comprising
   i. glycoprotein 1
   ii. glycoprotein 2
   iii. ginseng extract and
   iv. horsetail extract
      into cosmetic bases, it being preferred that the concentration of snow algae extract of 20% by weight, based on the total weight of the preparation, is not exceeded. Preference is also given to the incorporation of snow algae extract into cosmetic and/or dermatological bases where, during the incorporation, the temperature of 60° C. is not exceeded. Furthermore, it is preferred that, during the incorporation of snow algae extract into cosmetic and/or dermatological bases, temperatures of 50° to 60° C. must only prevail temporarily.

Also in accordance with the invention is the incorporation of snow algae extract and/or snow algae extract and one or more of the following substances:
d. collagen and/or a derivative
e. chitosan and/or acetylated chitosan with a degree of acetylation of about 50%
f. at least one glycosylaminoglycan and/or a derivative and at least one, preferably both, of the following ingredients:
f. at least one peptide which promotes cell growth,
g. a composition comprising
   v. glycoprotein 1
   vi. glycoprotein 2
   vii. ginseng extract and
   viii. horsetail extract
      into cosmetic bases, which lead to cosmetic products which remain on the skin, i.e. are applied and are not washed off again. Particular preference is given to those embodiments which serve to care for the skin.

Also in accordance with the invention is the incorporation of snow algae extract and/or snow algae extract and one or more of the following substances:
a. collagen and/or a derivative
b. chitosan and/or acetylated chitosan with a degree of acetylation of about 50%
c. at least one glycosylaminoglycan and/or a derivative and at least one, preferably both, of the following ingredients:
d. at least one peptide which promotes cell growth,
e. a composition containing
   i. glycoprotein 1
   ii. glycoprotein 2
   iii. ginseng extract and
   iv. horsetail extract
      into cosmetic bases which are rinsed off again following application to the skin, so-called rinse-off products. Furthermore, it is also possible to provide preparations according to the invention which serve for producing products which are used in hair cosmetics.

The teaching of the present invention also describes cosmetic and/or dermatological preparations which comprise snow algae extracts and one or more of the following substances:
a. collagen and/or a derivative
b. chitosan and/or acetylated chitosan with a degree of acetylation of about 50%
c. at least one glycosylaminoglycan and/or a derivative
   and at least one, preferably both, of the following ingredients:
d. at least one peptide which promotes cell growth,
e. a composition containing
   i. glycoprotein 1
   ii. glycoprotein 2
   iii. ginseng extract and
   iv. horsetail extract,
      where the combination of collagen (see a.), chitosan (see b.) and sodium chondroitin sulfate (see c.) is also referred to as Molecular Patch Complex (MPC). MPC is described in the applications WO 2010/086754 and US 2005/0249691. The disclosures relating to MPC from the aforementioned applications are hereby incorporated into this application.

The combination of glycoprotein 1 and 2 and ginseng and horsetail extract can be referred to as GPVE.

The substances under a. and b. may be of marine or synthetic origin.

The collagen from a. may preferably be selected from collagens which belong to types 1, 3, 4 and 5.

The chitosans under b. may preferably include chitosans which have a molecular weight of from about 80,000 to 15,000,000 g/mol. The chitosans can be obtained from insects and/or crustaceans.

The glycosylaminoglycans under c. may preferably include chondroitin-4-sulfate and/or chondroitin-6-sulfate.

The peptide or the peptides under d. may preferably be selected from peptides which are similar to cell growth factors or react like cell growth factors. Preference is given to a peptide with the INCI name Oligopeptide-21. This peptide is available for example from Caregen Co., Ltd., Korea under the trade name IDP-4. This oligopeptide-21 or at least part of oligopeptide-21 and indeed the part which binds to skin cell receptors, may be present. The peptide under d. is preferably in a form which prevents direct contact with a., b. and c. and e., if present. The particularly preferred form is the presence of d. in the form of a nanoemulsion.

The teaching regarding the substances listed above was disclosed in WO 2010/086754. WO 2010/086754 is hereby incorporated in its entirety into the disclosure of this application.

The cosmetic or dermatological preparations according to the invention can be present in the customary cosmetic and/or dermatological galenical preparation forms, preferably as gel, 0/W emulsion, W/O emulsion, W/O emulsion, microemulsion, cosmetic stick.

The cosmetic or dermatological products according to the invention can be present in the form of the customary cosmetic and/or dermatological product forms, preferably as aqueous surfactant-containing preparation, emulsion, ointment, cream, gel, powder, mask, foam preparation and aerosol preparation.

The preparations according to the invention are also suitable for producing sunscreen products. Sunscreen products are characterized by a content of UV filters which protect the skin against the harmful effects of sunlight.

Accordingly, within the context of the present invention, the preparations preferably comprise at least one UV-A, UV-B and/or broadband filter substance. The formulations may, although do not necessarily, optionally also comprise one or more organic and/or inorganic pigments as UV filter substances, which may be present in the water phase and/or the oil phase.

The preparations according to the present invention can in addition also advantageously be present in the form of so-called oil-free cosmetic emulsions which comprise a water phase and at least one UV filter substance that is liquid at room temperature as a further phase and which may in particular advantageously also be free from further oil components.

Within the context of the present invention, particularly advantageous UV filter substances that are liquid at room temperature are homomethyl salicylate (INCI: Homosalate), 2-ethylhexyl 2-cyano-3,3-diphenylacrylate (INCI: Octocrylene), 2-ethylhexyl 2-hydroxybenzoate (2-ethylhexyl salicylate, octylsalicylate, INCI: Ethylhexyl Salicylate) and esters of cinnamic acid, preferably 4-methoxycinnamic acid, 2-ethylhexyl ester (2-ethylhexyl 4-methoxycinnamate, INCI: Ethylhexyl Methoxycinnamate) and 4-methoxycinnamic acid isopentyl ester (isopentyl 4-methoxycinnamate, INCI: Isoamyl p-Methoxycinnamate), 3-(4-(2,2-bis ethoxycarbonylvinyl)phenoxy)propenyl) methoxysiloxane/dimethylsiloxane copolymer which is available for example under the trade name Parsol® SLX from Hoffmann La Roche.

Preferred inorganic pigments are metal oxides and/or other sparingly water-soluble or insoluble metal compounds, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and also the sulfate of barium (BaSO₄).

Within the context of the present invention, the pigments can advantageously also be used in the form of commercially available oily or aqueous predispersions. Dispersion auxiliaries and/or solubilization promoters can advantageously be added to these predispersions.

According to the invention, the pigments can advantageously be surface-treated (“coated”), in which case, for example, the intention is to form and/or retain a hydrophilic, amphiphilic or hydrophobic character. This surface treatment can consist in providing the pigments with a thin hydrophilic and/or hydrophobic inorganic and/or organic layer by methods known per se. Within the context of the present invention, the different surface coatings can also comprise water.

Suitable titanium dioxide particles and predispersions of titanium dioxide particles are available under the following trade names from the companies listed:

| Trade name | Coating | Manufacturer |
|---|---|---|
| MT-100TV | Aluminum | Tayca Corporation |
| | hydroxide/stearic acid | |
| MT-100Z | Aluminum hydroxide/ | Tayca Corporation |
| | stearic acid | |
| Eusolex T-2000 | Alumina/simethicone | Merck KgaA |
| Titandioxid T805 (Uvinul | Octyltrimethylsilane | Degussa |
| TiO₂) | | |
| Tioveil AQ 10PG | Alumina/silica | Solaveil/Uniquema |
| Eusolex T-aqua | Water/alumina/sodium | Merck |
| | metaphosphate | |

Within the context of the present invention, advantageous UV-A filter substances are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.

Within the context of the present invention, advantageous UV filter substances are also:
Phenylene-1,4-bis(2-benzimidazyl)-3,3′-5,5′-tetrasulfonic acid and its salts, particularly the corresponding sodium, potassium or triethanol ammonium salts, in particular the phenylene-1,4-bis(2-benzimidazyl)-3,3′-5,5′-tetrasulfonic acid bis-sodium salt with the INCI name Disodium Phenyl Dibenzimidazole Tetrasulfonate (CAS No.: 180898-37-7), which is available for example under the trade name Neo Heliopan AP from Symrise;
Salts of 2-phenylbenzimidazole-5-sulfonic acid, such as its sodium, potassium or its triethanol ammonium salt, and also the sulfonic acid itself with the INCI name Phenylbenzimidazole sulfonic acid (CAS No. 27503-81-7), which is available for example under the trade name Eusolex 232 from Merck or under Neo Heliopan Hydro from Symrise;
1,4-Di(2-oxo-10-sulfo-3-bornylidenemethyl)benzene (also: 3,3′-(1,4-phenylenedimethylene)bis(7,7-dimethyl-2-oxobicyclo[2.2.1]hept-1-ylmethane sulfonic acid) and salts thereof (particularly the corresponding 10-sulfato compounds, in particular the corresponding sodium, potassium or triethanolammonium salt), which is also referred to as benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor sulfonic acid (CAS No.: 90457-82-2) and is available for example under the trade name Mexoryl SX from Chimex;
Sulfonic acid derivatives of 3-benzylidenecamphor, such as e.g. 4-(2-oxo-3-bornylidenemethyl)benzene sulfonic acid, 2-methyl-5-(2-oxo-3-bornylidene-methyl)sulfonic acid and salts thereof.
Benzoxazole derivatives, such as e.g. 2,4-bis[5-1(dimethylpopryl)benzoxazol-2-yl-(4-phenyl)imino]-6-(2-ethylhexyl)imino-1,3,5-triazine with the CAS No. 288254-16-0, which is available from 3V Sigma under the trade name Uvasorb® K2A.
Hydroxybenzophenones, e.g. the 2-(4′-diethylamino-2′-hydroxybenzoyl)benzoic acid hexyl ester (also: aminobenzophenone), which is available under the trade name Uvinul A Plus from BASF.
Triazine derivatives, such as e.g. 2,4-bis{[4-(2-ethylhexyloxy)-2-hydroxyl]phenyl}-6-(4-methoxyphenyl)-1,3,5-triazine (INCI: Bis-Ethylhexyloxyphenol Methoxyphenyl Triazine), which is available under the trade name Tinosorb® S from CIBA-Chemikalien GmbH; dioctylbutylamidotriazone (INCI: Diethylhexyl Butamido Triazone), which is available under the trade name UVASORB HEB from Sigma 3V; tris(2-ethylhexyl) 4,4′,4″-(1,3,5-triazine-2,4,6-triyltriimino)trisbenzoate, also: 2,4,6-tris[anilino (p-carbo-2′-ethyl-1′-hexyloxy)]-1,3,5-triazine (INCI: Ethylhexyl Triazone), which is sold by BASF Aktiengesellschaft under the trade name UVINUL® T 150; 2-[4,6-bis(2,4-dimethylphenyl)-1,3,5-triazine-2-yl]-5-(octyloxy)phenol (CAS No.: 2725-22-6).
Benzotriazoles, such as e.g. 2,2′-methylene bis(6-2H-benzotriazol-2-yl)-4-(1,1,3,3-tetramethylbutyl) phenol) (INCI: Methylene Bis-Benztriazolyl Tetramethylbutylphenol), which is available e.g. under the trade name Tinosorb® M from CIBA-Chemikalien GmbH.
3-Benzylidenecamphor derivatives, preferably 3-(4-methylbenzylidene)camphor, 3-benzylidenecamphor;
4-Aminobenzoic acid derivatives, preferably 2-ethylhexyl 4-(dimethylamino)benzoate, amyl 4-(dimethylamino)benzoate;
Esters of benzalmalonic acid, preferably di(2-ethylhexyl) 4-methoxybenzalmalonate;
Esters of cinnamic acid, preferably 2-ethylhexyl 4-methoxycinnamate, isopentyl 4-methoxycinnamate;
Derivatives of benzophenone, preferably 2-hydroxy-4-methoxybenzophenone, 2-hydroxy-4-methoxy-4′-methylbenzophenone, 2,2′-dihydroxy-4-methoxybenzophenone, and
UV filters bonded to polymers
Ethylhexyl 2-cyano-3,3-diphenylacrylate (octocrylene), which is available from BASF under the name Uvinul^{Ò}. N 539 T.

Within the context of the present invention, particularly advantageous preparations which are characterized by a high or very high UV-A protection preferably also comprise, besides the filter substance(s) according to the invention, further UV-A and/or broadband filters, in particular dibenzoylmethane derivatives [for example 4-(tert-butyl)-4′-methoxydibenzoylmethane] and/or 2,4-bis{[4-(2-ethylhexyloxy)-2-hydroxy]phenyl}-6-(4-methoxyphenyl)-1,3,5-triazine and/or phenylene-1,4-bis(2-benzimidazyl)-3,3′-5,5′-tetrasulfonic acid bis-sodium salt, in each case individually or in any desired combinations with one another.

The list of specified UV filters which can be used within the context of the present invention is of course not intended to be limiting.

The total amount of filter substances is selected from the range from 0.1 to 30% by weight, preferably 0.5 to 10% by weight, in particular 1.0 to 6.0% by weight—in each case based on the total weight of the preparations—in order to provide cosmetic preparations which protect the hair and/or the skin from the entire range of ultraviolet radiation.

The cosmetic preparations according to the invention can comprise cosmetic auxiliaries as are customarily used in such preparations, e.g. preservatives, preservative aids, complexing agents, bactericides, perfumes, substances for increasing foaming, dyes, pigments which have a coloring effect, thickeners, moisturizing and/or humectant substances, fillers which improve the skin feel, fats, oils, waxes or other customary constituents of a cosmetic formulation such as alcohols, polyols, polymers, foam stabilizers, electrolytes, organic solvents or silicone derivatives.

Advantageous preservatives within the context of the present invention are for example iodopropyl butylcarbamates (e.g. those available under the trade names Glycacil-L, Glycacil-S from Lonza and/or Dekaben LMB from Jan Dekker), parabens (i.e. p-hydroxybenzoic acid alkyl esters, such as methyl-, ethyl-, propyl- and/or butylparaben), phenoxyethanol, ethanol, benzoic acid and the like. Usually, according to the invention, the preservative system further advantageously also comprises preservative aids, such as, for example, octoxyglycerol, glycine soya etc.

Advantageous complexing agents within the context of the present invention are, for example, EDTA, [S,S]-ethylenediaminedisuccinate (EDDS), which is available for example under the trade name Octaquest from Octel, pentasodium ethylenediaminetetramethylenephosphonate, which is available e.g. under the trade name Dequest 2046 from Monsanto and/or iminodisuccinic acid, which is available inter alia from Bayer AG under the trade names Iminodisuccinate VP OC 370 (ca. 30% strength solution) and Baypure CX 100 solid.

Particularly advantageous preparations are also obtained if antioxidants are used as additives or active ingredients. According to the invention, the preparations advantageously comprise one or more antioxidants. Favorable, but nevertheless optional antioxidants that can be used are all antioxidants that are suitable or customary for cosmetic applications.

Within the context of the present invention, water-soluble antioxidants can be used particularly advantageously, such as, for example, vitamins.

Preferred antioxidants are also vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant or the antioxidants, it is advantageous to select their particular concentrations from the range from 0.001 to 10% by weight, based on the total weight of the formulation.

If vitamin A or vitamin A derivatives or carotenes or derivatives thereof are the antioxidant or antioxidants, it is advantageous to select their respective concentrations from the range from 0.001 to 10% by weight, based on the total weight of the formulation.

Further active ingredients to be used advantageously within the context of the present invention are those which have a positive influence on the condition of the skin, such as in particular active ingredients for positively influencing ageing skin which reduce the formation of wrinkles and also existing wrinkles. In particular, bioquinones, in particular ubiquinone (Q10) and ubiquinol, folic acid and its derivatives (in particular tetrahydrofolic acid and dihydrofolic acid), niacin and its derivatives (in particular niacinamide), creatine and creatinine, carnitine, biotin, isoflavone, cardiolipin, lipoic acid, antifreezing proteins, hop and hop-malt extracts are advantageous. Agents which promote the restructuring of connective tissue, such as natural and/or synthetic isoflavonoids and also isoflavonoid-containing plant extracts—such as e.g. soya and clover extracts—can also be very readily used in the formulations according to the invention. It is also found that the formulations are particularly suitable for using active ingredients to support the skin functions in dry skin (such as for example vitamin C, biotin, carnitine, propionic acid, green tea extracts, eucalyptus oil, urea and mineral salts (such as e.g. NaCl, sea minerals), and also osmolytes (such as e.g. inositol, betaine, quaternary ammonium compounds)). In a similar way, the incorporation of active ingredients for alleviating and/or positively influencing irritative skin conditions, whether in the case of sensitive skin in general or skin irritated by noxae (UV light, chemicals), has proven to be advantageous. Here, mention is to be made of active ingredients such as sericosides, various extracts of licorice, licochalcones, in particular licochalcone A, silymarin, silyphos, dexpanthenol, inhibitors of the prostaglandin metabolism, in particular of the cyclooxygenase and of the leucotriene metabolism, in particular of 5-lipoxygenase, but also of the 5-lipoxygenase inhibitor protein, FLAP. In particular, creatine and creatinine are suitable active ingredients for setting up and/or renewing an (energy) depot, and to activate the repair of various cellular structures, in particular DNA. Also, the incorporation of pigmentation modulators has proven to be advantageous. Here, mention is to be made of active ingredients which reduce the pigmentation of the skin and thus lead to a cosmetically desired lightening of the skin and/or reduce the appearance of age spots and/or lighten existing age spots. By way of example, mention may be made of tyrosine sulfate, dioic acid (8-hexadecene-1,16-dicarboxylic acid), and lipoic acid and liponamide, various extracts of licorice, kojic acid, hydroquinone, arbutin, alpha-arbutin, deoxyarbutin, fruit acids, in particular alpha-hydroxy acids (AHAs), bearberry (Uvae ursi), ursolic acid, ascorbic acid, green tea extracts, aminoguanidine, pyridoxamine, niacinamide, inhibitors of the Proteinase Activated Receptors 2 (PAR-2). Particular preference is also given to formulations according to the invention which comprise further active ingredients which bring about an increased or more rapid tanning of the skin (Advanced Glycation End products (AGE), lipofuscins, nucleic acid oligonucleotides, purines and pyrimidines, NO-releasing substances), tyrosine and its derivatives (in particular N-acetyl tyrosine), phenylalanine and its derivatives (in particular n-acetyl phenylalanine), activators of the Proteinase Activated Receptors 2 (PAR-2), whether with or without the influence of UV light.

Formulations according to the invention which comprise e.g. known antiwrinkle active ingredients such as flavone glycosides (in particular α-glycosylrutin), coenzyme Q10, vitamin E and/or derivatives and the like, are particularly advantageously suitable for the prophylaxis and treatment of cosmetic or dermatological changes in the skin, as arise e.g. during skin ageing (such as for example dryness, roughness and formation of dryness wrinkles, itching, reduced refatting (e.g. after washing), visible vascular dilations (telangiectases, couperosis), flaccidity and formation of lines and wrinkles, local hyper-, hypo and mal-pigmentations (e.g. age spots), increased susceptibility to mechanical stress (e.g. cracking) and the like). Furthermore, they are advantageously suitable for counteracting the appearance of dry and/or rough skin.

Moisturizers is the term used to refer to substances or substance mixtures which give cosmetic preparations the property, following application and spreading on the skin surface, of reducing the release of moisture in the horny layer (also called transepidermal water loss (TEWL)) and/or of positively influencing the hydration of the horny layer.

Advantageous moisturizers within the context of the present invention are, for example, glycerol, lactic acid and/or lactates, in particular sodium lactate, butylene glycol, propylene glycol, biosaccharide gum-1, glycine soya, ethylhexyloxyglycerol, pyrrolidone carboxylic acid and urea.

The preparations according to the invention can furthermore comprise one or more substances which can be selected from amino acids, α-biotin, (NH₄)₆MO₇O₂₄, adenine, AlCl₃, biotin, CaCl₂, calcium pantothenate, choline chloride, CoCl₂, CrK(SO₄)₂, CuSO₄, D-Ca pantothenate, EDTA.Na₂-EDTA-Na₃, Fe(NO₃)₃, FeSO₄, folic acid, glucose, H₂SeO₃, HEPES, hypoxanthine, human insulin, KCl, linoleic acid, lipoic acid, MgCl₂, MnCl₂, MnSO₄, myo-inositol, Na₂HPO₄, Na₂SeO₃, Na₂SiO₃, NaCl, NaH₂PO₄, NaHCO₃, sodium pyruvate, sodium acetate, NH₄VO₃, NiCl₂, nicotinamide, phenyl red, polysorbate 80, putrescine, putrescine 2HCl, pyridoxine HCl, pyridoxal HCl, riboflavin, SnCl₂, thiamine HCl, thymidine, vitamin B₁₂, a ceramide, and ZnSO₄. The amino acids include L-alanine, L-arginine, L-asparagine, L-aspartic acid, L-cystein, L-cystine, glycine, L-glutamine, L-glutamic acid, L-histidine, L-isoleucine, L-leucine, L-lysine, L-methionine, L-phenylalanine, L-proline, L-serine, L-threonine, L-tryptophan, L-tyrosine, and L-valine. One or more amino acids can be selected. In the preparations according to the invention, the amino acids may be present for example as L-lysine and/or lysine HCl.

The preparations according to the invention can furthermore comprise additives of one or more cell culture media for skin cell culture. Examples are DMEM/HAM F12 (1:1) and/or MCDB 153.

The preparation can furthermore citrate buffer and/or Q10 and/or alpha-glycosylrutin and/or Zn orotate and/or carnitine and taurine and/or one or more alpha-hydroxy acids.

The oil phase of the preparations within the context of the present invention advantageously comprises nonpolar comprise, for example those which are selected from the group of branched and unbranched hydrocarbons and hydrocarbon waxes, in particular mineral oil, Vaseline (petrolatum), paraffin oil, squalane, polyolefins, hydrogenated polyisobutenes and isohexadecane. Among the polyolefins, polydecenes are the preferred substances.

The oil phase can also advantageously have a content of cyclic or linear silicone oils or consist entirely of such oils, although it is preferred to use an additional content of other oil phase components apart from the silicone oil or the silicone oils.

Silicone oils are synthetic compounds in which silicon atoms are linked via oxygen atoms in a chain-like and/or net-like manner, and the remaining valences of the silicon are saturated by hydrocarbon radicals (in most cases methyl groups, less frequently ethyl, propyl, phenyl groups etc.). Systematically, the silicone oils are referred to as polyorganosiloxanes. The methyl-substituted polyorganosiloxanes, which represent the most important compounds of this group in terms of amount and are characterized by the following structural formula

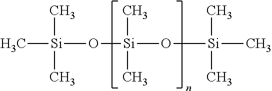

are also referred to as polydimethylsiloxane or Dimethicone (INCI). Dimethicones come in various chain lengths and with different molecular weights.

According to the invention, it is advantageous to use cyclomethicone (octamethylcyclotetrasiloxane) as silicone oil. However, other silicone oils are also to be used advantageously within the context of the present invention, for example hexamethylcyclotrisiloxane, polydimethylsiloxane, poly(methylphenylsiloxane).

Particularly advantageous polyorganosiloxanes within the context of the present invention are for example dimethylpolysiloxanes[poly(dimethylsiloxane)], which are available for example under the trade names Abil 10 to 10 000 from Th. Goldschmidt Also advantageous are phenylmethylpolysiloxanes (INCI: Phenyl Dimethicone, Phenyl Trimethicone), cyclic silicones (octamethylcyclotetrasiloxane and decamethylcyclopentasiloxane), which are referred to in accordance with INCI also as Cyclomethicones, amino-modified silicones (INCI: Amodimethicones) and silicone waxes, e.g. polysiloxane-polyalkylene copolymers (INCI: Stearyl Dimethicone and Cetyl Dimethicone) and dialkoxydimethylpolysiloxanes (Stearoxy Dimethicone and Behenoxy Stearyl Dimethicone), which are available as various Abil wax grades from Th. Goldschmidt However, other silicone oils are also to be used advantageously within the context of the present invention, for example cetyldimethicone, hexamethylcyclotrisiloxane, polydimethylsiloxane, poly(methylphenylsiloxane).

The preparations according to the present invention can also advantageously comprise one or more substances from the following group of siloxane elastomers, for example in order to increase the water resistance and/or the sun protection factor of the products:
(a) siloxane elastomers which contain the units R₂SiO and RSiO_{1.5} and/or R₃SiO_{0.5} and/or SiO₂,
   where the individual radicals R are in each case, independently of one another, hydrogen, C₁₋₂₄-alkyl (such as for example methyl, ethyl, propyl) or aryl (such as for example phenyl or tolyl), alkenyl (such as for example vinyl) and the weight ratio of the units R₂SiO to RSiO_{1.5} is chosen from the range from 1:1 to 30:1;
(b) siloxane elastomers which are insoluble and swellable in silicone oil and which are obtainable by the addition reaction of an organopolysiloxane (1) which contains silicon-bonded hydrogen with an organopolysiloxane (2) which contains unsaturated aliphatic groups, where the quantitative fractions used are selected such that the amount of hydrogen in the organopolysiloxane (1) or the unsaturated aliphatic groups in the organopolysiloxane (2) is
   in the range from 1 to 20 mol % if the organopolysiloxane is not cyclic and
   in the range from 1 to 50 mol % if the organopolysiloxane is cyclic.

Within the context of the present invention, the siloxane elastomer or elastomers are advantageously present in the form of spherical powders or in the form of gels.

Siloxane elastomers present in the form of spherical powders that are advantageous according to the invention are those with the INCI name Dimethicone/Vinyl Dimethicone Crosspolymer, for example that available from DOW CORNING under the trade names DOW CORNING 9506 Powder.

It is particularly preferred if the siloxane elastomer is used in combination with oils of hydrocarbons of animal and/or vegetable origin, synthetic oils, synthetic esters, synthetic ethers or mixtures thereof.

It is very particularly preferred if the siloxane elastomer is used in combination with unbranched silicone oils or cyclic silicone oils, or mixtures thereof, that are liquid or pasty at room temperature. Organopolysiloxane elastomers with the INCI name Dimethicone/Polysilicone-11, very particularly the Gransil grades GCM, GCM-5, DMG-6, CSE Gel, PM Gel, LTX, ININ Gel, AM-18 Gel and/or DMCM-5 available from Grant Industries Inc. are especially advantageous.

According to the invention, the customary packaging as are used in cosmetics or dermatology are suitable. For certain preparation forms, however, it may be advantageous to keep different components of the preparation according to the invention separate and only bring them together shortly before use. It is then particularly advantageous within the context of the present invention to provide packagings which have a two-chamber technology.

The so-called bag-on-valve systems, consisting of a valve which is hermetically sealed with a film bag, for example, are advantageous. The film bag is optimally multiply with at least one or more barrier layers. The bag valve is hermetically sealed prior to filling with an aerosol container (aluminum, tinplate or plastic).

In the so-called bag-in-can system, the bag is located in an aerosol container and, upon closing, is joined to the valve and the aerosol container.

Packagings of this type are described in DE-20301831, EP-972923, EP-1061006 or EP-586295, which are hereby incorporated into the disclosure of the present invention.

The container is preferably supplied with environmentally friendly propellants, such as nitrogen or noble gases, with the propellant being located between bag and closed aerosol container, whereas the cosmetic is located in the bag.

The mode of function of the bag-in-can systems or bag-on-valve systems can be described as follows: the propellant surrounds the bag filled with the cosmetic. When the nozzle, the spray or application head of the system, is pressed down, the pressure on the bag causes the contents to emerge in varying amounts depending on the diameter of the nozzle. In this connection, the preparation located in the bag has to be matched to the packaging in order to achieve the correct discharge for a pregiven can pressure as regards the viscosity.

An advantage of two-chamber technology (bag-in-can systems, bag-on-valve systems) is that no contamination and thus also no air can penetrate into the pressurized bag.

The dispensing head optimally includes, at the exit opening, a closure mechanism which reduces possible contamination of the product between valve opening and exit opening.

The self-closing dispensing head can be described in detail e.g. as follows:

By actuating the applicator, an aerosol valve is opened, the product flows through a channel to the exit opening. Upon passing through the channel, the elastic element (e.g. made of a thermoplastic elastomer (TPE), polyethylene, polypropylene, polyoxymethylene (POM)) expands, which keeps the channel closed as far as the exit opening in the untriggered state. The prerequisite for this is a higher pressure inside the can relative to the ambient atmosphere.

Upon closing the aerosol valve, the pressure between aerosol valve and exit opening is reduced to the point of complete sealing. By reducing the pressure, the elastic element drops into the product-conveying channel, and the remaining fill material is applied until the point of complete closure of the channel.

In this way, contamination of the remaining fill material in the dispensing head by the ambient atmosphere is prevented until the aerosol valve is next opened.

The self-closing dispensing head can also be detailed e.g. as follows:

By actuating the applicator, an aerosol valve is opened, an elastic membrane springs opposite to the product stream. A closure pin inseparably joined to the membrane follows this movement, and the exit opening/ring die is opened.

Upon closing the aerosol valve, the product stream decreases. As a result of the pressure drop, membrane and closure pin move into their starting position and the exit opening/ring die is closed.

This principle also optimally protects the fill material in the head.

The effectiveness of the snow algae extracts was demonstrated in several test series.

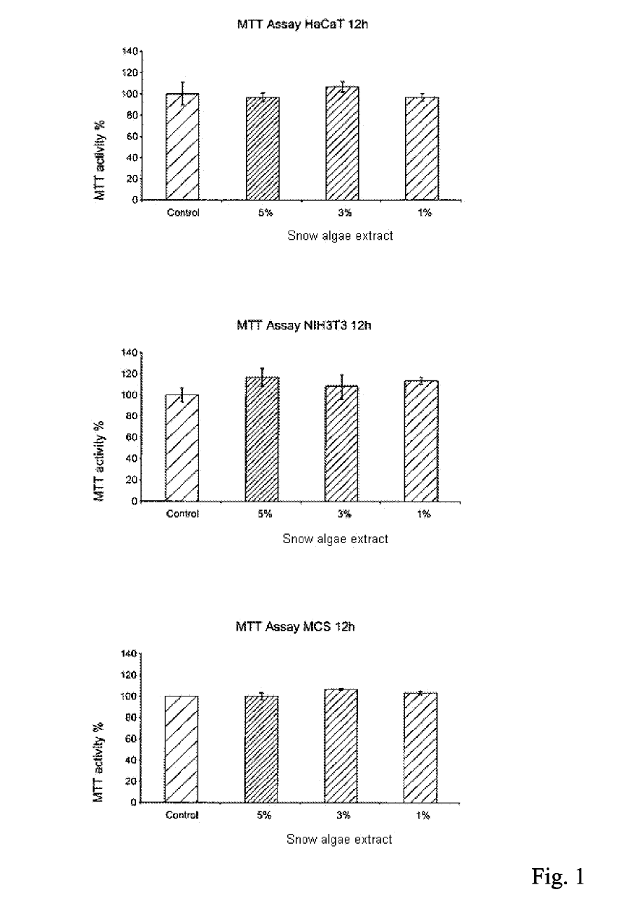

In a cytotoxicity test, the MTT assay, the compatibility of the snow algae extracts was demonstrated. For this purpose, keratinocytes were incubated with different concentrations of snow algae extract for 12 hours. The cell vitality was then determined with the MTT assay. In the MTT assay, a mitochondrial dehydrogenase in living cells cleaves the tetrazolium ring of the pale yellow substance MTT (3-(4,5-dimethylthiazolyl)-2,5-diphenyltetrazolium bromide). Dark blue formazan crystals are formed which accumulate in intact cells. The cells are lysed and the crystals released. The amount of crystals formed is determined photometrically at 595 nm and is a direct measure of the number of intact cells. FIG. 1 shows the results of the treatment of HaCaT cells (spontaneously immortalized keratinocytes), NIH3T3 fibroblasts and mesenchymal stem cells (MSC) with snow algae extracts and the determination of the cell vitality. No toxic effect arises during a snow algae extract treatment of these cell types.

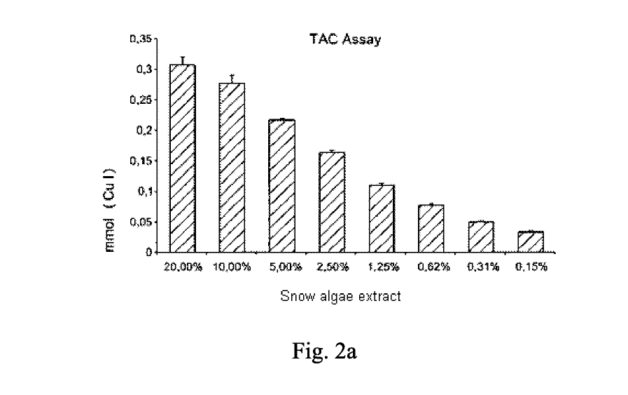

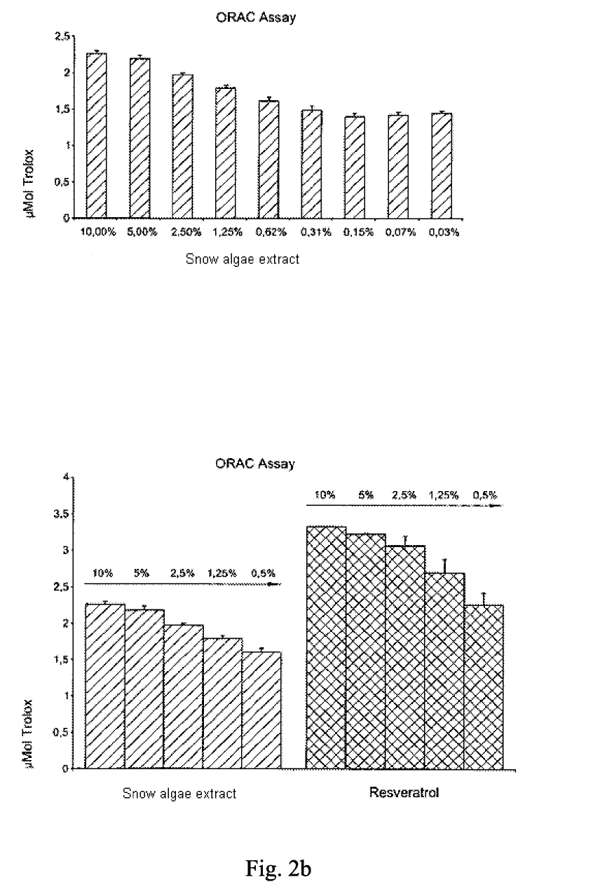

The antioxidative effectiveness of the snow algae extracts was determined with two different test methods. The measurement of the antioxidative capacity is based on a redox reaction between a test substance, here snow algae extract, and copper II (Cu(II)). If the test substance has a reducing effect, Cu(II) is converted to Cu(I). Cu(I) is detected by the addition of bathocuproine. The photometric measurement takes place at 490 nm. The results of this test are shown in FIG. 2a. The reducing power of the snow algae extract at different concentrations becomes evident.

The capacity of a substance to absorb oxygen radicals is determined in the ORAC (oxygen radical absorbance capacity) test. The ability of a test substance to inhibit the oxidation of a fluorophore, in most cases fluorescein, by a strong oxidizing agent is determined. The strong oxidizing agent is 2,2′-azobis(2-amidino propane). The progress of the reaction is monitored by measurements at 535 nm. The standard used is the substance Trolox. Trolox is a water-soluble vitamin E derivative and has a marked antioxidative capacity and is therefore used as a reference substance. The standard curve is obtained by plotting the different Trolox concentrations against the average value of the area determinations of two measurements for each concentration. For the evaluation, the net area under the curve (AUC) of the samples and of the standard solutions in different concentrations is determined. The net area values are obtained from the difference of the sample area values or standard area values and the area value under the zero value. The zero value is determined without the addition of sample or standard. The ORAC values of the samples are given as gmol of Trolox equivalents per liter. The results of the ORAC test for snow algae extract are shown in FIG. 2b. The upper part of the diagram shows that snow algae extract has a good antioxidative power down to low concentration ranges (0.03%). In order to support this data, comparisons with Resveratrol were carried out. Resveratrol is a phytoalexin and belongs to the polyphenols. It was isolated for the first time in 1963 from Japanese knotweed. This substance has strong antioxidative properties. The results in the lower part of FIG. 2b exhibit a good antioxidative effectiveness for snow algae extract, but do not quite pertain the effectiveness of Resveratrol.

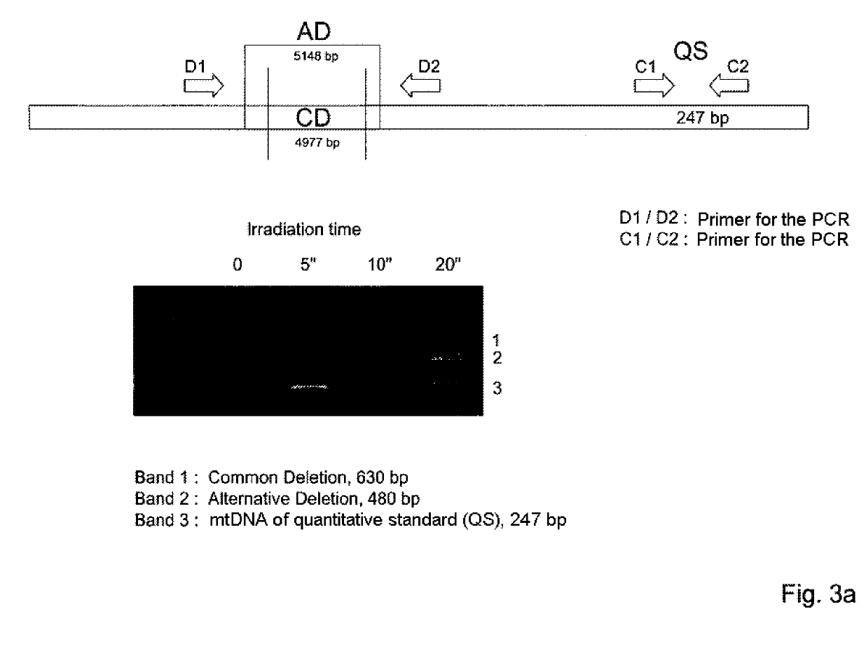

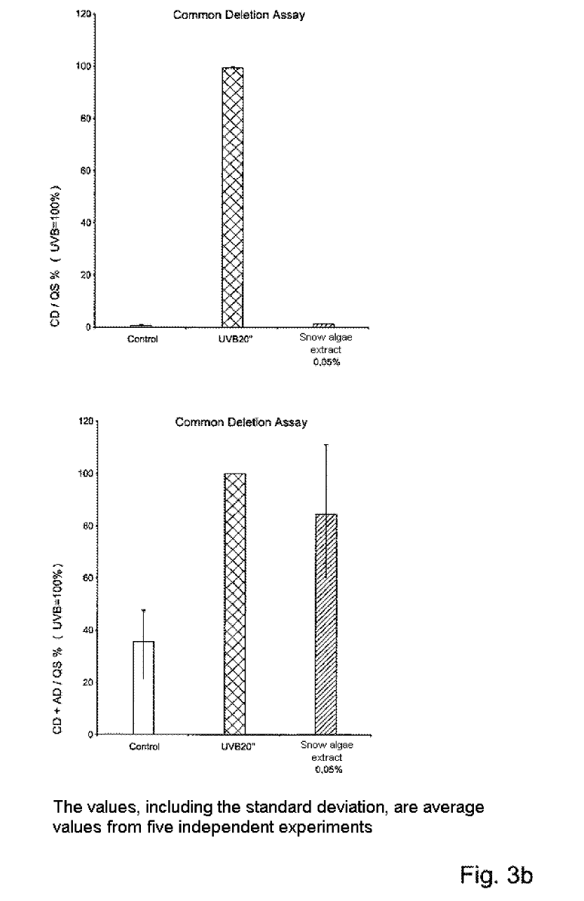

Mutations of mitochondrial DNA (mtDNA) occur during the normal ageing process. The most common mutation is a deletion of 4.977 bp, also called the common deletion. This mutation can be detected using a test with the help of PCR (common deletion assay). Keratinocytes were cultured and treated for 48 hours with snow algae extract (0.05%). After replacing the cell culture medium with PBS solution, the cells were irradiated with UVB light (1.5 mJ/cm²). Incubation was then carried out for one hour in cell culture medium at 37° C. These cells were harvested, and the DNA was extracted using a QiagenKit. For the PCR, 2-5 ng of DNA were used. Two deletions were detected, the common deletion (CD), which comprises 4977 bp, in a 630 bp band and the Alternative Deletion (AD), which comprises 5148 bp, in a 480 bp band (FIG. 3a). As the standard, a 247 bp fragment was amplified. With the help of the intensity of the deletion bands, expressed as a ratio of CD band to the standard band, it is possible to determine the protection against damage by UV light that is achieved by the snow algae extract (FIG. 3b above). In the second part of the figure (FIG. 3b at the bottom), the AD band is included in the evaluation. The intensity of the AD band is given as a ratio of the sum of CD band and AD band to the standard band. It is clear that snow algae extract protects against damage by UV light to a great extent.

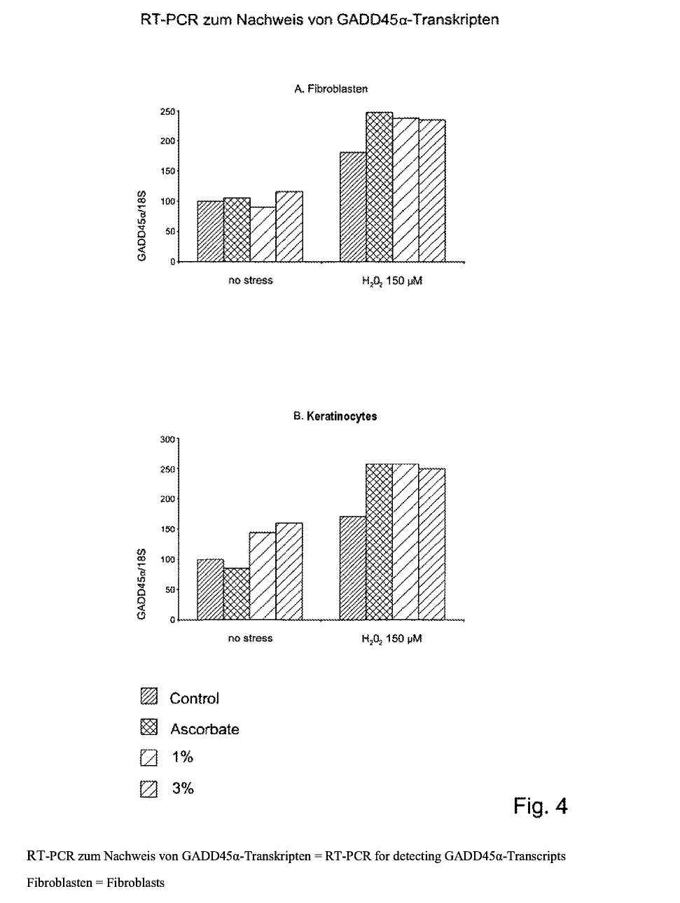

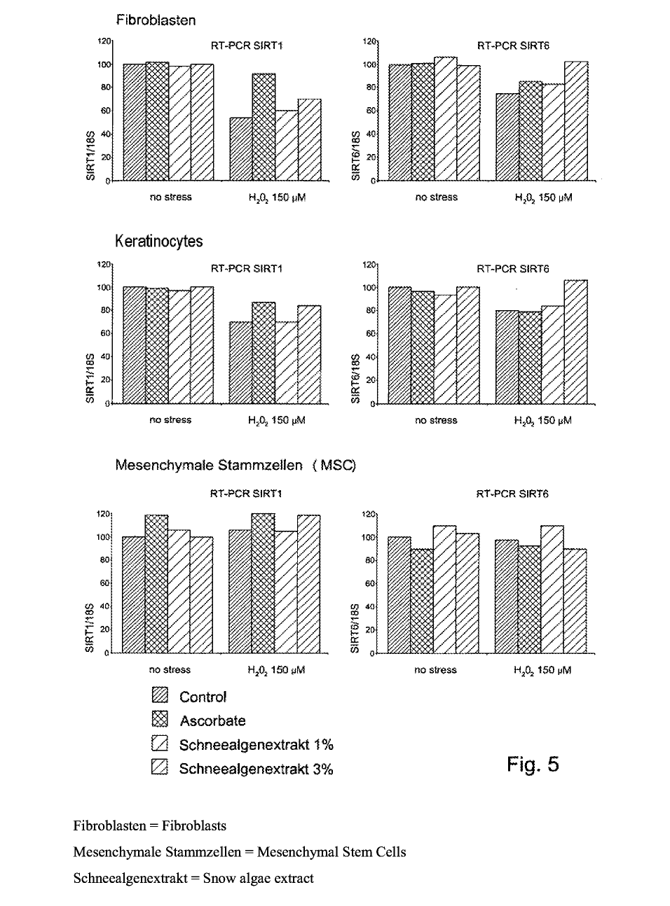

Every living eukaryotic cell has a cell core in which the genetic information is localized in the chromosomes. Before each cell division, the genetic information is copied. Transcription of the genetic information leads to the formation of mRNA, which is translated into the corresponding proteins (protein biosynthesis). Many environmental factors, primarily modern ones, mean stress for the cells. To protect themselves, they stop growing. This growth stop is characterized by an increase in the transcription rate of the GADD45A gene. GADD45A is the abbreviation for growth arrest and DNA-damage-inducible protein alpha. The transcription rate of the GADD45A gene is also increased after a treatment with DNA-harming substances. The transcription rate of GADD45A can be demonstrated with the help of PCR reactions (FIG. 4). Fibroblasts and keratinocytes were treated with 1% or 3% snow algae extract or ascorbate for two hours. Control cells were not treated. The cells were then subjected to a stress factor, and specifically in the form of H₂O₂ (150 μM). At different intervals, the cells were harvested and the RNA was isolated. The RNA was used as a starting point to synthesize cDNA, which served as a matrix for the PCR. The primers used for the PCR were those which are specific for the GADD45A transcript. The PCR products were separated in an agarose gel and quantified using the Geliance 200 Imaging System (Perkin Elmer). The amplification band of the analyzed transcription was normalized with the amplification band of a standard 18S transcript. The values obtained in this way had been given in FIG. 3 as percentages. The untreated controls corresponded to 100% values. In fibroblasts, no increase in the GADD45A transcripts is observed under control conditions. After the stress treatment, more GADD45A transcripts can be detected in the cells treated with snow algae extract or with ascorbate than in the control cells. This means that, under stress conditions, snow algae extract in a concentration of 3% brings about an increased rate in GADD45A transcripts (58%) and thus boosts the protection for the cells. In the case of the keratinocytes, the non-stressed, untreated cells also exhibited an increased transcription rate of GADD45A after the action of snow algae extract. The transcription is increased considerably (79%, at 3% snow algae extract) under stress conditions. This shows that snow algae extract is suitable for activating protection mechanisms in skin cells and specifically in particular under stress conditions.

In a further test series, sirtuin transcripts were detected. Sirtuins are enzymes which deacetylate histone proteins. As a result, lysine radicals are released and the nitrogen atom in the amine radical of this amino acid condenses to a greater extent with the DNA. As a result, transcription processes are disturbed or prevented. This leads to a so-called “gene-silencing”. In addition, sirtuins also have ADP-ribosyltransferase activity. Besides the histones, a series of other proteins is also modified. An influence on the cell ageing was demonstrated. The experiments to detect sirtuin transcripts were carried out analogously to the experiments for detecting the GADD45A transcripts. However, primers for detecting sirtuin transcripts in the PCR reaction were specific for sirtuin 1 (Sirt 1) or sirtuin 6 (Sirt 6). The evaluation and preparation was carried out in the same way as described for GADD45A. Under stress conditions, triggered by H₂O₂, the transcription rate increases in fibroblasts which have been incubated with snow algae extract of sirtuin 1 by 18% and sirtuin 6 by 28% compared to the untreated cells. In keratinocytes, the transcription rate of sirtuin 1 increases by 14% and of sirtuin 6 by 27% as a result of the effect of snow algae extract compared to untreated cells. In mesenchymal stem cells, no significant reduction in the sirtuin gene expression was observed under stress conditions by H₂O₂ (up to 450 μM). Overall, the snow algae extract triggered a slight increase in the sirtuin gene expression irrespective of the stress effect. As a result of the effect of snow algae extract it was possible to demonstrate in three different cell types that there is an influence on the sirtuin gene expression.

The examples below serve to illustrate the present invention without limiting it. The numerical values in the examples are percentages by weight, based on the total weight of the respective preparations.

### EXAMPLE 1

### 2—Phase Product

For use, 7 ml of phase 1 are mixed with 1 ml of phase 2.

Phase 1 (clear serum)

| Formula (INCI) | % w/w |
|---|---|
| WATER (AQUA) | 70.9439 |
| ALCOHOL DENAT. | 6.000 |
| METHYL GLUCETH-20 | 4.000 |
| BIS-PEG-18 METHYL ETHER DIMETHYL SILANE | 4.000 |
| PROPYLENE GLYCOL | 3.000 |
| BUTYLENE GLYCOL | 3.000 |
| GLYCERIN | 2.388 |
| PEG-12 DIMETHICONE | 2.000 |
| PANTHENOL | 0.855 |
| BENZOPHENONE-4 | 0.600 |
| PEG-40 HYDROGENATED CASTOR OIL | 0.463 |
| AMMONIUM ACRYLOYLDIMETHYLTAURATE/VP | 0.380 |
| COPOLYMER | |
| ARGININE | 0.350 |
| DISODIUM EDTA | 0.305 |
| XANTHAN GUM | 0.180 |
| DISODIUM ADENOSINE TRIPHOSPHATE | 0.068 |
| SODIUM HYALURONATE | 0.050 |
| ETHYLHEXYL METHOXYCINNAMATE | 0.025 |
| BUTYL METHOXYDIBENZOYLMETHANE | 0.024 |
| PPG-26-BUTETH-26 | 0.023 |
| ALCOHOL | 0.023 |
| LECITHIN | 0.023 |
| ETHYLHEXYL SALICYLATE | 0.015 |
| CARICA PAPAYA (PAPAYA) FRUIT EXTRACT | 0.013 |
| ALGIN | 0.013 |
| LEPIDIUM SATIVUM SPROUT EXTRACT | 0.003 |
| GLYCINE SOJA (SOYBEAN) OIL | 0.002 |
| HYDROGENATED LECITHIN | 0.002 |
| POLYSORBATE 80 | 0.001 |
| OLIGOPEPTIDE-21 | 0.001 |
| SODIUM OLEATE | 0.000 |
| FRAGRANCE (PARFUM) | 0.140 |
| LINALOOL | 0.026 |
| MENZYL ALCOHOL | 0.021 |
| HYDROXYCITRONELLAL | 0.005 |
| CITRONELLOL | 0.005 |
| ALPHA-ISOMETHYL IONONE | 0.005 |
| AMYL CINNAMAL | 0.004 |
| HEXYL CINNAMAL | 0.003 |
| EVERNIA FURFURACEA (TREEMOSS) EXTRACT | 0.003 |
| BENZYL BENZOATE | 0.002 |
| GERANIOL | 0.002 |
| BUTYLPHENYL METHYLPROPIONAL | 0.002 |
| PHENOXYETHANOL | 0.732 |
| METHYLPARABEN | 0.300 |
| | 100.0000 |

pH adjusted to 5.4-5.7
Phase 2 (blueish cream gel)

| Formula (INCI) | % w/w |
|---|---|
| WATER (AQUA) | 73.9798 |
| CYCLOPENTASILOXANE | 4.6880 |
| PEG-12 DIMETHICONE | 3.0000 |
| POLYDECENE | 2.0000 |
| PENTYLENE GLYCOL | 2.0000 |
| GLYCERIN | 1.5595 |
| ISOMALT | 1.5540 |
| ETHYLHEXYL METHOXYCINNAMATE | 1.2500 |
| BUTYL METHOXYDIBENZOYLMETHANE | 1.2000 |
| PEG-40 HYDROGENATED CASTOR OIL | 1.1500 |
| PPG-26-BUTETH-26 | 1.1500 |
| VP/HEXADECENE COPOLYMER | 1.0000 |
| ETHYLHEXYL SALICYLATE | 0.7500 |
| GLYCOPROTEINS | 0.6770 |
| AMMONIUM | 0.5700 |
| ACRYLOYLDIMETHYLTAURATE/BEHENETH-25 | |
| METHACRYLATE CROSSPOLYMER | |
| HYDROXYETHYL ACRYLATE/SODIUM | 0.3700 |
| ACRYLOYLDIMETHYL TAURATE COPOLYMER | |
| DIMETHICONE | 0.3200 |
| ALCOHOL | 0.3130 |
| DIMETHICONOL | 0.3000 |
| SQUALANE | 0.2500 |
| POLYSILICONE-11 | 0.2400 |
| BUTYLENE GLYCOL | 0.1600 |
| PANAX GINSENG ROOT EXTRACT | 0.1008 |
| POLOXAMER 188 | 0.1000 |
| DISODIUM EDTA | 0.1000 |
| BUTYL STEARATE | 0.0834 |
| SOLUBLE COLLAGEN | 0.0683 |
| EQUISETUM ARVENSE (HORSETAIL) EXTRACT | 0.0504 |
| TOCOPHEROL | 0.0500 |
| POLYSORBATE 60 | 0.0500 |
| GENIPA AMERICANA FRUIT EXTRACT | 0.0476 |
| CHITOSAN | 0.0183 |
| CHLAMYDOMONAS EXTRACT | 0.0168 |
| SODIUM CHLORIDE | 0.0107 |
| GLYCINE | 0.0089 |
| VITIS VINIFERA (GRAPE) SEED EXTRACT | 0.0085 |
| LECITHIN | 0.0080 |
| DECYL GLUCOSIDE | 0.0080 |
| SODIUM CHONDROITIN SULFATE | 0.0077 |
| GLUCOSE | 0.0075 |
| VITIS VINIFERA (GRAPE) FRUIT CELL EXTRACT | 0.0067 |
| LYSINE HCL | 0.0024 |
| CAPRYLYL GLYCOL | 0.0020 |
| LACTIC ACID | 0.0017 |
| THREONINE | 0.0016 |
| ARGININE | 0.0014 |
| POTASSIUM CHLORIDE | 0.0007 |
| HISTIDINE | 0.0007 |
| SERINE | 0.0007 |
| HEXYLENE GLYCOL | 0.0006 |
| CALCIUM CHLORIDE | 0.0004 |
| TRYPTOPHAN | 0.0003 |
| MAGNESIUM SULFATE | 0.0003 |
| SODIUM PHOSPHATE | 0.0002 |
| FOLIC ACID | 0.0001 |
| CALCIUM PANTOTHENATE | 0.0001 |
| PHENOXYETHANOL | 0.5681 |
| METHYLPARABEN | 0.1650 |
| SODIUM BENZOATE | 0.0050 |
| PROPYLPARABEN | 0.0033 |
| ETHYLPARABEN | 0.0033 |
| SODIUM DEHYDROACETATE | 0.0025 |
| BUTYLPARABEN | 0.0017 |
| ISOBUTYLPARABEN | 0.0017 |
| POTASSIUM SORBATE | 0.0008 |
| EXT. VIOLET 2 | 0.0019 |
| RED 33 | 0.0004 |
| BLUE 1 | 0.0002 |
| | 100.0000 |

pH adjusted to 4.7-5.2

### EXAMPLE 2

| | O/W Cream | |
|---|---|---|
| | 1 | 2 |
| Formula (INCI) | % w/w | % w/w |
| WATER (AQUA) | 54.3830 | 54.2884 |
| GLYCERIN | 5.3350 | 5.3350 |
| MYRISTYL LACTATE | 5.0000 | 5.0000 |
| CYCLOPENTASILOXANE | 3.7700 | 3.7700 |
| BUTYROSPERMUM PARKII (SHEA BUTTER) | 3.0800 | 3.0800 |
| GLYCERYL STEARATE CITRATE | 3.0000 | 3.0000 |
| HYDROGENATED COCO-GLYCERIDES | 2.5000 | 2.5000 |
| PENTYLENE GLYCOL | 2.2000 | 2.2000 |
| PROPYLHEPTYL CAPRYLATE | 2.0000 | 2.0000 |
| OCTYLDODECANOL | 2.0000 | 2.0000 |
| CETEARYL ALCOHOL | 2.0000 | 2.0000 |
| POLY(GLYCOL ADIPATE)/BIS- | 1.5000 | 1.5000 |
| HYDROXYETHOXYPROPYL DIMETHICONE | | |
| COPOLYMER | | |
| PEG-30 STEARATE | 1.1000 | 1.1000 |
| CYCLOHEXASILOXANE | 1.0500 | 1.0500 |
| BUTYL METHOXYDIBENZOYLMETHANE | 1.0000 | 1.0000 |
| LAUROYL LYSINE | 1.0000 | 1.0000 |
| AMMONIUM | 0.7600 | 0.7600 |
| ACRYLOYLDIMETHYLTAURATE/VP | | |
| COPOLYMER | | |
| PANTHENOL | 0.7500 | 0.7500 |
| GLYCERYL DIBEHENATE | 0.6600 | 0.6600 |
| PROPYLENE GLYCOL | 0.6250 | 0.6250 |
| TOCOPHERYL ACETATE | 0.6000 | 0.6000 |
| FUMARIA OFFICINALIS | 0.5000 | 0.5000 |
| FLOWER/LEAF/STEM EXTRACT | | |
| CITRUS MEDICA LIMONUM (LEMON) | 0.5000 | 0.5000 |
| FRUIT EXTRACT | | |
| TRIBEHENIN | 0.3600 | 0.3600 |
| CAPRYLIC/CAPRIC TRIGLYCERIDE | 0.3200 | 0.3200 |
| GALACTOARABINAN | 0.3000 | 0.3000 |
| BUTYLENE GLYCOL | 0.2619 | 0.2619 |
| HYDROGENATED LECITHIN | 0.2420 | 0.2420 |
| XANTHAN GUM | 0.2390 | 0.2390 |
| POLOXAMER 188 | 0.2000 | 0.2000 |
| CAFFEINE | 0.2000 | 0.2000 |
| GLYCERYL BEHENATE | 0.1800 | 0.1840 |
| ARGININE | 0.1704 | 0.1800 |
| FUMARIC ACID | 0.1250 | 0.1704 |
| POLYSILICONE-11 | 0.1050 | 0.1250 |
| DISODIUM PHOSPHATE | 0.1000 | 0.1050 |
| ISOMALT | 0.0920 | 0.1000 |
| DIMETHICONOL | 0.0750 | 0.0750 |
| HIBISCUS ABELMOSCHUS SEED EXTRACT | 0.0700 | 0.0700 |
| DISODIUM EDTA | 0.0540 | 0.0540 |
| ETHYLHEXYLGLYCERIN | 0.0505 | 0.0505 |
| LECITHIN | 0.0402 | 0.0403 |
| CITRIC ACID | 0.0400 | 0.0400 |
| BHT | 0.0300 | 0.0300 |
| SODIUM HYALURONATE | 0.0300 | 0.0300 |
| RUTIN | 0.0250 | 0.0250 |
| SQUALANE | 0.0200 | 0.0200 |
| ALCOHOL | 0.0200 | 0.0200 |
| SOLUBLE COLLAGEN | 0.0171 | 0.0171 |
| POLYSORBATE 80 | 0.0110 | 0.0110 |
| LEPIDIUM SATIVUM SPROUT EXTRACT | 0.0048 | 0.0048 |
| CHITOSAN | 0.0046 | 0.0046 |
| CERAMIDE 3 | 0.0040 | 0.0040 |
| SODIUM CHLORIDE | 0.0027 | 0.0027 |
| CHLAMYDOMONAS EXTRACT | 0.0020 | 0.0040 |
| GLYCINE SOJA (SOYBEAN) OIL | 0.0020 | 0.0020 |
| SODIUM CHONDROITIN SULFATE | 0.0019 | 0.0019 |
| GLUCOSE | 0.0019 | 0.0019 |
| LYSINE HCL | 0.0006 | 0.0006 |
| OLIGOPEPTIDE-21 | 0.0005 | 0.0005 |
| THREONINE | 0.0004 | 0.0004 |
| POTASSIUM CHLORIDE | 0.0002 | 0.0004 |
| HISTIDINE | 0.0002 | 0.0002 |
| SERINE | 0.0002 | 0.0002 |
| LACTIC ACID | 0.0002 | 0.0002 |
| SODIUM OLEATE | 0.0002 | 0.0002 |
| SODIUM PHOSPHATE | 0.0001 | 0.0001 |
| TRYPTOPHAN | 0.0001 | 0.0001 |
| MAGNESIUM SULFATE | 0.0001 | 0.0001 |
| CALCIUM CHLORIDE | 0.0001 | 0.0001 |
| GLYCINE | 0.0001 | 0.0001 |
| CALCIUM PANTOTHENATE | 0.0001 | 0.0001 |
| FOLIC ACID | 0.0001 | 0.0001 |
| FRAGRANCE (PERFUME) | 0.2810 | 0.2810 |
| BUTYLPHENYL METHYLPROPIONAL | 0.0210 | 0.0210 |
| BENZYL SALICYLATE | 0.0210 | 0.0210 |
| HEXYL CINNAMAL | 0.0158 | 0.0158 |
| BENZYL ALCOHOL | 0.0035 | 0.0035 |
| GERANIOL | 0.0025 | 0.0025 |
| LINALOOL | 0.0023 | 0.0023 |
| HYDROXYCITRONELLAL | 0.0021 | 0.0021 |
| LIMONENE | 0.0007 | 0.0007 |
| ISOEUGENOL | 0.0001 | 0.0001 |
| PHENOXYETHANOL | 0.5261 | 0.5261 |
| METHYLPARABEN | 0.2021 | 0.2021 |
| PROPYLPARABEN | 0.2008 | 0.2008 |
| BUTYLPARABEN | 0.0017 | 0.0017 |
| ISOBUTYLPARABEN | 0.0004 | 0.0006 |
| SODIUM BENZOATE | 0.0003 | 0.0004 |
| ETHYLPARABEN | 0.0002 | 0.0002 |
| YELLOW 6 | 0.0002 | 0.0002 |
| | 100.0000 | 100.0000 |

pH adjusted to 5.8-6.2

### EXAMPLE 3

### O/W Cream

| Formula (INCI) | % w/w |
|---|---|
| WATER (AQUA) | 61.6860 |
| GLYCERIN | 5.4312 |
| POLYMETHYLSILSESQUIOXANE | 4.8000 |
| TRIDECYL STEARATE | 4.2500 |
| CYCLOPENTASILOXANE | 3.2900 |
| HDI/TRIMETHYLOL HEXYLLACTONE | 3.2000 |
| CROSSPOLYMER | |
| PETROLATUM | 2.0000 |
| SORBITAN TRISTEARATE | 2.0000 |
| CETYL PEG/PPG-10/1 DIMETHICONE | 2.0000 |
| HYDROXYETHYL ACRYLATE/SODIUM | 1.8000 |
| ACRYLOYLDIMETHYL TAURATE COPOLYMER | |
| DIMETHICONE | 1.4470 |
| POLYISOBUTENE | 0.9000 |
| CETYL ALCOHOL | 0.5258 |
| STEARYL ALCOHOL | 0.5237 |
| POLYSILICONE-11 | 0.5160 |
| CAPRYLYL GLYCOL | 0.5000 |
| PEG-40 STEARATE | 0.5000 |
| SORBITAN STEARATE | 0.5000 |
| SODIUM CHLORIDE | 0.5000 |
| DIMETHICONE/DIVINYLDIMETHICONE/ | 0.4300 |
| SILSESQUIOXANE CROSSPOLYMER | |
| HYDROGENATED COCO-GLYCERIDES | 0.4300 |
| ISODODECANE | 0.2500 |
| PROPYLENE GLYCOL | 0.2193 |
| STEAROXYMETHICONE/DIMETHICONE COPOLYMER | 0.2000 |
| HEXYLENE GLYCOL | 0.1500 |
| PEG-7 TRIMETHYLOLPROPANE COCONUT ETHER | 0.1500 |
| TOCOPHERYL ACETATE | 0.1215 |
| OCTADECENEDIOIC ACID | 0.1000 |
| ISOMALT | 0.0920 |
| ISOHEXADECANE | 0.0860 |
| POLYSORBATE 80 | 0.0774 |
| THALASSIOSIRA PSEUDONANA EXTRACT | 0.0500 |
| AMMONIUM POLYACRYLOYLDIMETHYL TAURATE | 0.0430 |
| GLYCOPROTEINS | 0.0403 |
| CETYL PALMITATE | 0.0400 |
| POLYSORBATE 20 | 0.0323 |
| GLYCERYL STEARATE | 0.0301 |
| SORBITAN OLIVATE | 0.0300 |
| SORBITAN PALMITATE | 0.0300 |
| CENTELLA ASIATICA EXTRACT | 0.0250 |
| BOSWELLIA SERRATA GUM EXTRACT | 0.0250 |
| HONEY (MEL) | 0.0250 |
| TREMELLA FUCIFORMIS (MUSHROOM) EXTRACT | 0.0220 |
| BEHENYL ALCOHOL | 0.0215 |
| TUBER MAGNATUM EXTRACT | 0.0200 |
| RESVERATROL | 0.0200 |
| LARIX SIBIRICA WOOD EXTRACT | 0.0165 |
| PALMITIC ACID | 0.0151 |
| SODIUM HYALURONATE | 0.0120 |
| STEARIC ACID | 0.0108 |
| LECITHIN | 0.0105 |
| DEXTRAN | 0.0104 |
| HYDROXYPROLINE | 0.0104 |
| RETINYL PALMITATE | 0.0099 |
| PANAX GINSENG ROOT EXTRACT | 0.0090 |
| GLYCINE | 0.0066 |
| CARBOMER | 0.0060 |
| CAVIAR EXTRACT | 0.0058 |
| POLYGONUM CUSPIDATUM ROOT EXTRACT | 0.0050 |
| VITIS VINIFERA (GRAPE) VINE EXTRACT | 0.0038 |
| EQUISETUM ARVENSE (HORSETAIL) EXTRACT | 0.0030 |
| PROLINE | 0.0020 |
| CHLAMYDOMONAS EXTRACT | 0.0020 |
| ACRYLATES COPOLYMER | 0.0015 |
| LEPIDIUM SATIVUM SPROUT EXTRACT | 0.0012 |
| SORBITOL | 0.0008 |
| POLYMETHYL METHACRYLATE | 0.0007 |
| NELUMBO NUCIFERA FLOWER EXTRACT | 0.0005 |
| JASMINUM OFFICINALE (JASMINE) EXTRACT | 0.0005 |
| PALMITOYL HEPTAPEPTIDE-5 | 0.0005 |
| SODIUM ASCORBYL PHOSPHATE | 0.0003 |
| LACTIC ACID | 0.0002 |
| TRICAPRYLIN | 0.0002 |
| TOCOPHEROL | 0.0001 |
| GLYCYRRHIZA GLABRA (LICORICE) ROOT EXTRACT | 0.0001 |
| XANTHAN GUM | 0.0001 |
| ISOOCTANOYL TETRAPEPTIDE-25 | 0.0001 |
| DISODIUM EDTA | 0.0001 |
| GLYCERYL CAPRYLATE | 0.0001 |
| FRAGRANCE (PARFUM) | 0.1756 |
| LINALOOL | 0.0868 |
| CITRONELLOL | 0.0221 |
| GERANIOL | 0.0063 |
| LIMONENE | 0.0042 |
| EUGENOL | 0.0015 |
| BENZYL BENZOATE | 0.0013 |
| BENZYL SALICYLATE | 0.0012 |
| FARNESOL | 0.0005 |
| CITRAL | 0.0004 |
| PHENOXYETHANOL | 0.4194 |
| SODIUM BENZOATE | 0.0045 |
| SODIUM DEHYDROACETATE | 0.0002 |
| POTASSIUM SORBATE | 0.0001 |
| | 100.0000 |

pH adjusted to 6.0-6.4

### EXAMPLE 4

### Serum

| Formula (INCI) | % w/w |
|---|---|
| WATER (AQUA) | 72.9901 |
| GLYCERIN | 6.0116 |
| ALCOHOL DENAT. | 3.0003 |
| CYCLOPENTASILOXANE | 2.9883 |
| BUTYLENE GLYCOL | 2.3206 |
| DIMETHICONE | 2.3202 |
| ASCORBYL GLUCOSIDE | 2.0002 |
| BIOSACCHARIDE GUM-1 | 1.4807 |
| HDI/TRIMETHYLOL HEXYLLACTONE | 1.4702 |
| CROSSPOLYMER | |
| SODIUM CITRATE | 0.5001 |
| INULIN LAURYL CARBAMATE | 0.5001 |
| AMINOMETHYL PROPANOL | 0.475 |
| AMMONIUM | 0.3801 |
| ACRYLOYLDIMETHYLTAURATE/BEHENETH-25 | |
| METHACRYLATE CROSSPOLYMER | |
| POLYACRYLAMIDE | 0.3501 |
| ISOMALT | 0.2741 |
| SODIUM POLYACRYLATE | 0.2501 |
| POLYSILICONE-11 | 0.24 |
| VEGETABLE (OLUS) OIL | 0.2 |
| POLOXAMER 188 | 0.2 |
| PROPYLENE GLYCOL | 0.1913 |
| C13-C14 ISOPARAFFIN | 0.17 |
| SODIUM STEAROYL LACTYLATE | 0.14 |
| CALCIUM ALUMINUM BOROSILICATE | 0.1352 |
| XANTHAN GUM | 0.1051 |
| SODIUM LACTATE | 0.1 |
| TOCOPHERYL ACETATE | 0.1 |
| OCTADECENEDIOIC ACID | 0.1 |
| SQUALANE | 0.0941 |
| LAURETH-7 | 0.08 |
| CETYL ALCOHOL | 0.06 |
| HIBISCUS ABELMOSCHUS SEED EXTRACT | 0.0393 |
| SILICA | 0.039 |
| LECITHIN | 0.0205 |
| ALCOHOL | 0.02 |
| CHOLESTERYL NONANOATE | 0.0175 |
| CHOLESTERYL OLEATE | 0.0165 |
| CHOLESTERYL STEARATE | 0.016 |
| SODIUM CARBOXYMETHYL BETA-GLUCAN | 0.016 |
| GLYCINE SOJA (SOYBEAN) STEROLS | 0.01 |
| GLYCYRRHIZA GLABRA (LICORICE) ROOT EXTRACT | 0.01 |
| GLYCOPROTEINS | 0.0081 |
| DECYL GLUCOSIDE | 0.008 |
| CHLAMYDOMONAS EXTRACT | 0.006 |
| SOLANUM LYCOPERSICUM (TOMATO) FRUIT | 0.006 |
| EXTRACT | |
| BUTYL STEARATE | 0.0056 |
| SOLUBLE COLLAGEN | 0.0046 |
| CAVIAR EXTRACT | 0.0046 |
| DISODIUM EDTA | 0.004 |
| CARNOSINE | 0.004 |
| LEPIDIUM SATIVUM SPROUT EXTRACT | 0.0024 |
| OCTYLDODECANOL | 0.0021 |
| CAPRYLYL GLYCOL | 0.002 |
| HYDROGENATED LECITHIN | 0.002 |
| GLYCINE SOJA (SOYBEAN) OIL | 0.002 |
| RESVERATROL | 0.002 |
| TIN OXIDE | 0.0018 |
| PANAX GINSENG ROOT EXTRACT | 0.0013 |
| CAMELLIA SINENSIS LEAF EXTRACT | 0.0013 |
| CHITOSAN | 0.0012 |
| LARIX SIBIRICA WOOD EXTRACT | 0.001 |
| POLYSORBATE 80 | 0.001 |
| SODIUM CHLORIDE | 0.0007 |
| LEONTOPODIUM ALPINUM FLOWER/LEAF EXTRACT | 0.0007 |
| HEXYLENE GLYCOL | 0.0006 |
| EQUISETUM ARVENSE (HORSETAIL) EXTRACT | 0.0006 |
| LACTIC ACID | 0.0006 |
| SODIUM CHONDROITIN SULFATE | 0.0005 |
| GLUCOSE | 0.0005 |
| POLYGONUM CUSPIDATUM ROOT EXTRACT | 0.0005 |
| SODIUM HYALURONATE | 0.0005 |
| OLIGOPEPTIDE-34 | 0.0005 |
| ACETYL OCTAPEPTIDE-3 | 0.0003 |
| LYSINE HCL | 0.0002 |
| SODIUM OLEATE | 0.0002 |
| NELUMBO NUCIFERA FLOWER EXTRACT | 0.0001 |
| ARGININE | 0.0001 |
| THREONINE | 0.0001 |
| HYDROXYPROLINE | 0.0001 |
| DEXTRAN | 0.0001 |
| POTASSIUM CHLORIDE | 0.0001 |
| GLYCINE | 0.0001 |
| ISOOCTANOYL TETRAPEPTIDE-25 | 0.0001 |
| HISTIDINE | 0.0001 |
| SERINE | 0.0001 |
| CALCIUM PANTOTHENATE | 0.0001 |
| FOLIC ACID | 0.0001 |
| TRYPTOPHAN | 0.0001 |
| MAGNESIUM SULFATE | 0.0001 |
| CALCIUM CHLORIDE | 0.0001 |
| FRAGRANCE (PARFUM) | 0.0293 |
| LINALOOL | 0.0145 |
| CITRONELLOL | 0.0037 |
| GERANIOL | 0.0011 |
| LIMONENE | 0.0007 |
| EUGENOL | 0.0003 |
| BENZYL BENZOATE | 0.0002 |
| BENZYL SALICYLATE | 0.0002 |
| CITRAL | 0.0001 |
| FARNESOL | 0.0001 |
| METHYLPARABEN | 0.3049 |
| PHENOXYETHANOL | 0.0749 |
| ETHYLPARABEN | 0.0013 |
| BUTYLPARABEN | 0.0013 |
| SODIUM BENZOATE | 0.001 |
| PROPYLPARABEN | 0.0006 |
| ISOPROPYLPARABEN | 0.0004 |
| ISOBUTYLPARABEN | 0.0002 |
| TITANIUM DIOXIDE | 0.054 |
| | 100.0000 |

pH adjusted to 6.0-6.4

1.-22. (canceled)

23. A cosmetic or dermatological preparation for protecting skin, wherein the preparation comprises snow algae extract.

24. The preparation of claim 23, wherein the preparation further comprises isomalt, lecithin, sodium benzoate, lactic acid, and water.

25. The preparation of claim 23, wherein the preparation comprises from 0.0001% to 99% by weight of snow algae extract, based on a total weight of the preparation.

26. The preparation of claim 23, wherein the preparation comprises from 0.0005% to 20% by weight of snow algae extract.

27. The preparation of claim 23, wherein the preparation comprises from 0.001% to 10% by weight of snow algae extract.

28. The preparation of claim 23, wherein the preparation further comprises (i) one or more of collagen and/or a derivative thereof, (ii) chitosan and/or acetylated chitosan having a degree of acetylation of about 50%, (iii) at least one glycosylaminoglycan and/or a derivative thereof, and (iv) at least one of (a) a peptide which promotes cell growth and (b) a composition comprising glycoprotein 1, glycoprotein 2, ginseng extract, and horsetail extract.

29. The preparation of claim 28, wherein both (iv)(a) and (iv)(b) are present.

30. The preparation of claim 28, wherein the preparation further comprises isomalt, lecithin, sodium benzoate, lactic acid, and water.

31. The preparation of claim 28, wherein the preparation comprises from 0.0005% to 20% by weight of snow algae extract, based on a total weight of the preparation.

32. The preparation of claim 29, wherein the preparation comprises from 0.001% to 10% by weight of snow algae extract, based on a total weight of the preparation.

33. The preparation of claim 30, wherein the preparation comprises from 0.0005% to 20% by weight of snow algae extract, based on a total weight of the preparation.

34. The preparation of claim 30, wherein the preparation comprises from 0.001% to 10% by weight of snow algae extract, based on a total weight of the preparation.

35. A method of protecting skin, wherein the method comprises applying to skin in need thereof the preparation of claim 23.

36. The method of claim 35, wherein skin is protected against modern stresses.

37. The method of claim 35, wherein skin is protected against effects of free radicals.

38. The method of claim 35, wherein skin is protected against effects of UV light.

39. A method of increasing the longevity of skin cells, wherein the method comprises applying to skin in need thereof the preparation of claim 23.

40. The method of claim 39, wherein intrinsic and/or extrinsic skin ageing is counteracted.

41. A method of protecting skin, wherein the method comprises applying to skin in need thereof the preparation of claim 28.

42. A method of protecting skin, wherein the method comprises applying to skin in need thereof the preparation of claim 34.
